(19) United States
(12) Patent Application Publication
(10) Pub. No.: US 2020/0147369 A1
(43) Pub. Date: May 14, 2020

(54) METHODS AND DEVICES UTILIZING NON-IONIZING PULSED ELECTRIC FIELDS TO TREAT OR MODIFY BACTERIA IN THE BODY

(71) Applicant: GOVERMENT OF THE UNITED STATES AS REPRESENTED BY THE SECRETARY OF THE AIR FORCE, Wright-Patterson AFB, OH (US)

(72) Inventors: Stacey L. Martens, Converse, TX (US); Bennett L. Ibey, San Antonio, TX (US)

(21) Appl. No.: 16/184,122

(22) Filed: Nov. 8, 2018

(51) Int. Cl. A61N 1/20

(52) U.S. Cl. CPC A61N 1/205

(57) ABSTRACT

An ingestible capsule, a medical probe and method provide for in-vivo treatment of bacterial cells in the human body. A capsule body or a medical probe has at least two electrodes, a power supply, and a controller. The controller controls the delivery of non-ionizing nanosecond pulsed electric fields (nsPEF) to targeted bacteria within the human body for an exposure time and at an exposure location, initiating death in the bacterial cells.

### CROSS-REFERENCE TO RELATED APPLICATIONS

This application claims the benefit of priority under 35 U.S.C. § 119(e) to U.S. Provisional Application Ser. No. 62/675,204 entitled “METHODS AND DEVICES UTILIZING NON-IONIZING PULSED ELECTRIC FIELDS TO TREAT OR MODIFY BACTERIA IN THE BODY,” [Docket AFD-1687P] filed 23 May 2018, the contents of which are incorporated herein by reference in its entirety.

### ORIGIN OF THE INVENTION

The invention described herein was made by employees of the United States Government and may be manufactured and used by or for the Government of the United States of America for governmental purposes without the payment of any royalties thereon or therefore.

### BACKGROUND OF THE INVENTION

Examination into the microbiome as well as combatting drug resistant bacterial strains is a top priority for government and civilian sectors alike. From a military perspective, preventing disease or non-battle illness (DNBI) is a continuing challenge for warfighter readiness. According to a 2006 study investigating care provided during Operation Iraqi Freedom between the years of 2003-04, dysentery (a Gastrointestinal, bacterial induced ailment) was the second highest cause for hospital admittance, in DNBI cases.

Existing treatment methods for bacterial induced illnesses include the use of antibiotics that target and interfere with one of five methods in the cell synthesis process. However, due to random mutagenesis and the fast replication rate of bacteria, antibiotic resistant strains have emerged. The pharmaceutical approach to treating bacterial infections has immensely accelerated the rate of the emergence of antibiotic resistant strains. This is because antibiotics prevent growth of strains that do not have a mutated genetic code; this leaves only the genetically modified, resistant strains to grow.

Therefore, a device that is non-pharmaceutical in nature and capable of targeting both the parent strains and mutagenic strains of bacteria has a considerable advantage over antibiotics.

### BRIEF SUMMARY

In one aspect, the present disclosure provides an ingestible capsule for in-vivo treatment of bacterial cells in the human body. The ingestible capsule includes a capsule body carrying at least two electrodes, a power supply, and a controller. The controller, coupled to the power supply and the at least two electrodes, is configured to control the delivery of non-ionizing nanosecond pulsed electric fields (nsPEF) to targeted bacteria within the human body for an exposure time and at an exposure location.

In another aspect, the present disclosure provides a medical probe for in-vivo treatment of bacterial cells in the human body. The medical probe includes a scope carrying at least two electrodes at a distal end thereof, a power supply, and a controller. The controller, coupled to the power supply and the at least two electrodes, is configured to control the delivery of non-ionizing nanosecond pulsed electric fields (nsPEF) to targeted bacteria within the human body for an exposure time and at an exposure location.

In an additional aspect, the present disclosure provides a method for in-vivo treatment of bacterial cells in the human body. The method includes positioning at least two electrodes, coupled to a power supply, within the gastrointestinal tract. The method includes controlling the delivery of non-ionizing nanosecond pulsed electric fields (nsPEF) to targeted bacteria within the human body for an exposure time and at an exposure location to create electropores in membranes of the bacteria cells to initiate death in the bacterial cells.

The above summary contains simplifications, generalizations and omissions of detail and is not intended as a comprehensive description of the claimed subject matter but, rather, is intended to provide a brief overview of some of the functionality associated therewith. Other systems, methods, functionality, features and advantages of the claimed subject matter will be or will become apparent to one with skill in the art upon examination of the following figures and detailed written description.

### BRIEF DESCRIPTION OF THE DRAWINGS

The description of the illustrative embodiments can be read in conjunction with the accompanying figures. It will be appreciated that for simplicity and clarity of illustration, elements illustrated in the figures have not necessarily been drawn to scale. For example, the dimensions of some of the elements are exaggerated relative to other elements. Embodiments incorporating teachings of the present disclosure are shown and described with respect to the figures presented herein, in which:

FIG. 1 illustrates a functional block diagram of a small capsule that moves through a gastrointestinal (GI) tract of a person exposing the wall of GI organs (stomach, intestines and colon) to small electric pulses that do not harm body cells, according to one or more embodiments;

FIG. 2 illustrates an isometric view of an electrode scope, according to one or more embodiments;

FIG. 3 illustrates a perspective view of an exposure system used in an experimental demonstration of the present innovation, according to one or more embodiments;

FIG. 4 illustrates three graphical representations of thermal response, according to one or more embodiments;

FIG. 5 illustrates two graphical representations of L. acidophilus nsPEF and thermal response viability curves, according to one or more embodiments; and

FIG. 6 illustrates K. pneumoniae nsPEF and thermal response viability curves, according to one or more embodiments; and

FIG. 7 illustrates a flow diagram of a method 700 for in-vivo treatment of bacterial cells in the human body, according to one or more embodiments.

### DETAILED DESCRIPTION OF THE INVENTION

Methods and devices are described herein to combat bacterial induced ailments by utilizing a medical device that produces directed energy (e.g. a non-ionizing electrical pulse) to create pores in bacterial plasma membranes/cell walls and trigger cellular death. This innovative approach will be non-pharmaceutical, non-invasive and can target both susceptible and resistant strains of bacteria.

A purpose of this invention is to modulate bacterial growth within the Gastrointestinal (GI) System by targeted exposure of electrical pulses.

The embodiments of the invention aim to address two problems. The first problem is the time it takes to get a patient back to a healthy status (i.e., getting a patient healthier faster). The second problem is solving microbiome research gaps. In 2014, Executive Order 13676—Combatting Antibiotic-Resistant Bacteria was issued due to the growing concern over the emergence of antibiotic resistant microbes. This Executive Order pushed for a concerted effort to detect, prevent and control antibiotic resistance. While there has been a strong scientific effort aimed at determining the extent that our microbiomes influence our health there is a lack of methods to control for these types of bacterial infections. This invention will fill this research gap.

This invention could be used in clinical medical settings to treat a diagnosis of poor microbiome population and/or changes induced in the microbiome by medical treatment or diet. Additionally, this invention could be used in clinical and academic research settings.

The advantage to using electrical pulses as a vehicle to combat bacterial infections versus the current treatment method of antibiotics is through the differences in their modes-of-action. Each specific antibiotic targets a select moment in the cell synthesis process. Based on morphological differences in the physical structure of various types of bacteria, an antibiotic mode-of-action may limit the type of bacteria in which it can affect. To the contrary, electrical pulses can target all bacterium because it does not interfere with the cell synthesis process. Instead, electrical pulses create small pores in the cell wall and/or plasma membrane. These pores allow for an influx of extracellular fluid which will cause the cell to swell and eventually lyse, killing the bacterium. Advantages to this process include: the ability to target all bacterial types and the ability to combat those bacterial cell lines that have evolved or will evolve to evade current antibiotic treatments.

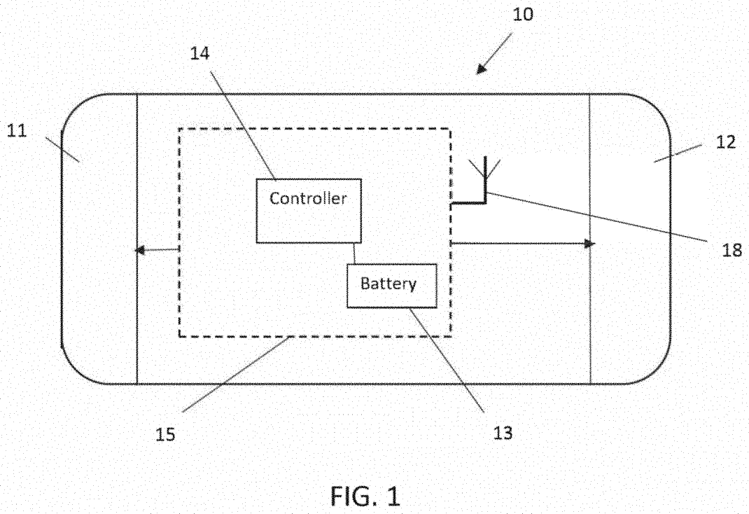

Referring to FIG. 1, an embodiment of the invention includes the use of a small capsule 10 that moves through the GI tract exposing the wall of GI organs (stomach, intestines and colon) to small electric pulses that do not harm body cells. The capsule 10 will be swallowed and then will move through the digestive track naturally. This device will include two electrode ends 11, 12 with a small battery 13 and controller 14 that can allow for exposure to the organs as it moves through the GI tract. The location and pulsing sequence may be controlled, monitored or updated via an external controller/receiver (e.g. computer or smart device) using wireless communications (e.g. RF, Bluetooth, NFC, etc.) via antenna 18.

Embodiments may use a small circuit board 15 that will take current from the battery 13 and send it to electrode poles 11, 12 on the capsule 10. These poles 11, 12 may be randomized to alternate the exposure of electric field (specifically the anode) to both sides of the intestine as the capsule 10 moves through the GI tract. The controller 14 may be a microcontroller or processor mounted to the circuit board 15 and may have a timing program that provides a precise exposure regiment at a GI location specific to the native environment for the bacteria of interest.

The capsule 10 may have exposed electrodes made of a biocompatible conductive material (e.g., tungsten) that interfaces with the biological environment. The remainder of the capsule 10 may be coated with a nonconductive material (e.g., polytetrafluoroethylene (PTFE) or Teflon) to isolate the two electrodes. The capsule 10 may use a single battery 13 and be disposed of after use to avoid the necessity to sterilize the device between uses. The small radiative antenna 18 connected to, or integrated on, the circuit board 15 will allow for communication and local tracking of the device as it passes through the GI tract.

A patient with a GI bacterial infection may be given the capsule 10 by a medical professional who has predetermined what type of infection is causing the distress. This information will be pertinent to help the provider determine the correct exposure parameters to deliver to the patient, as discussed below.

Prior to the patient ingesting the capsule, it should be tuned to deliver the appropriate electrical dosage to create pores in the targeted bacterial type and deliver the exposure at the correct time interval within the GI tract. Removal of the capsule 10 from the body will be as easy as waiting for the patient to excrete it through a bowel movement. This embodiment of the invention may be utilized as a single use device.

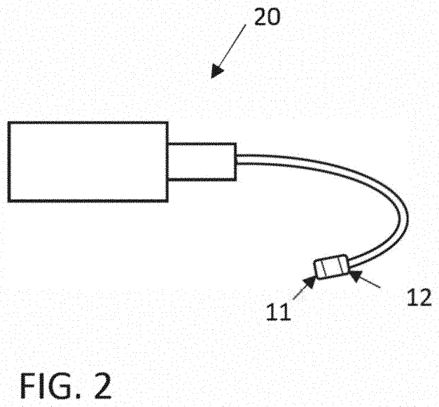

Referring additionally to FIG. 2, embodiments may also include the use of an electrode scope 20 during a colonoscopy to apply pulses directly to areas of the lower GI tract or through the esophagus. This embodiment could be sterilized and may be used only in a setting (e.g., a hospital) under medical supervision. The electrode ends 11, 12 may be coaxial or sealed multi-electrodes that can deliver electric fields a short distance from the end of the scope 20. Angled probes may also be constructed to allow for more precise targeting. The addition of a camera for endoscopic direct targeting by a physician is also a possibility.

Commercial industries have used pulsed electric field (PEF) technology as a technique to sterilize food, process waste water and disinfect topical burns from bacteria. Issues arise due to the thermal deposition produced by the combination of long pulse widths (micro-milliseconds), high repetition rates and large voltages. However, pulse generators capable of producing shorter pulse widths (nanoseconds) with field strengths as high as 300 kV/cm will be used in the present embodiments. Nanosecond pulsed electric fields (nsPEF) are advantageous to their micro-millisecond counterparts because they generate less heat while still generating high field strengths.

Exposing eukaryotic cells to nsPEF has shown that nsPEF can initiate cell death. However, little was known regarding the mechanism(s) of PEF cell inactivation. Current theories suggest that PEF creates electropores in the cell membrane and that electropore duration ranges from short-lived to irreversible electroporation depending on electric field intensity and pulse width. The present embodiments operate under the theory that nsPEF can also be utilized to initiate death in bacterial cells, specifically gram-negative species that reside in the gastrointestinal (GI) microbiome of Homo sapiens. Tests included exposing gram+/−bacteria to varying nsPEF parameters in vitro and measuring viability. Initial results suggest that: 1) nsPEF can be utilized to initiate death in GI bacteria; and, 2) gram negative species are more sensitive to the effects of nsPEF.

Cell Lines: Lactobacillus acidophilus (ATCC 4357) and Klebsiella pneumoniae (ATCC 10031) lyophilized pellets were purchased from ATCC and propagated according to manufacturer's protocol. Stocks were preserved by aliquoting bacterial broth with 50% glycerol (1:1) into 1.5 mL tubes and frozen at −80° C.

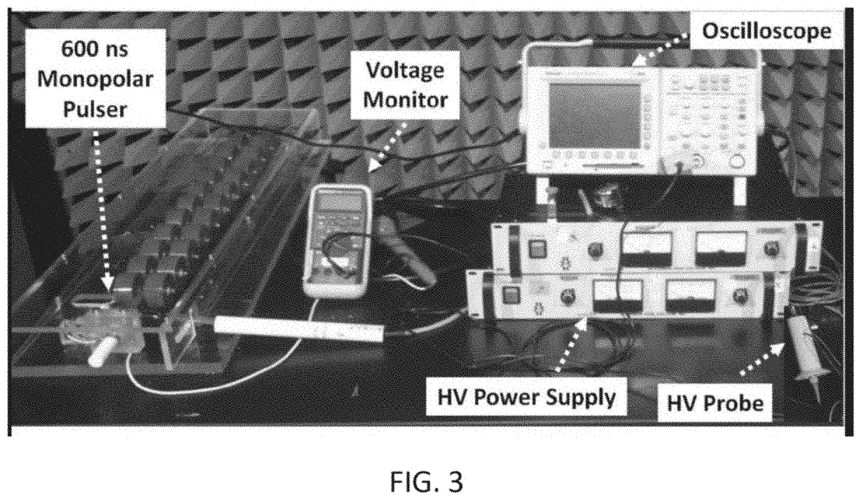

FIG. illustrates a perspective view of an exposure system used in an experimental demonstration of the present innovation. The Marx bank capacitor system was used to generate the 600 ns monopolar pulse (FIG. 3). A Tektronix high voltage probe connected to a high-speed oscilloscope (TDS 30504B) was used to measure the delivered pulse.

Cell Culture: Per ATCC recommendations, growth media was purchased from Becton, Dickson and Company (BD). De Man, Rogosa and Sharpe (MRS) broth (BD 288210) or Nutrient broth (BD 213000) were utilized to culture L. acidophilus and K. pneumoniae, respectively. Overnight (o/n) cultures were initiated by inoculating 1 vial from the −80° C. stock into 50 mls of MRS broth or Nutrient broth and incubated at 37° C. o/n. Stationary L. acidophilus cultures were incubated at elevated carbon dioxide levels using a BD GasPak 150 system. K pneumoniae cultures were incubated aerobically and shaken at 200 rpms. The following morning the optical density (O.D) of a 100 μl aliquot was read in a BioTek Synergy HTX spectrophotometer set to a wavelength of 600 nm. To initiate experiments, a fresh 50 mls of growth media was inoculated with enough o/n culture to provide a starting O.D. value of 0.05. Cultures were returned to their respective incubators and allowed to grow to log phase (approx. 4 hours).

Exposure & Viability Assay: Cells were prepared for nsPEF and thermal response exposures by following the “Culture conditions and preparation of bacterial suspensions” (section 1.1-1.8) of Molecular Probes Live/Dead BacLight Viability Kit (L7012). Cell concentration was determined as previously stated and adjusted to provide a final concentration of 1.5×10⁸ cells/mL.

nsPEF: A 400 μl cell suspension (cells and 0.85% sodium chloride [NaCl]) was pipetted into a 2 mm electroporation cuvette from VWR international and exposed to a random series of: 1, 5, 10, 100 or 1000 pulses with a 600-ns pulse width at 13.5, 18.5 or 23.5 kV/cm and a 1 Hz repetition rate. Sham exposures were performed by loading the cuvettes with cells and placing it into the pulser (8 minutes), but no pulse was delivered. All exposures were run in triplicate.

nsPEF Induced Temperature change: 400 μl of 0.85% NaCl solution was aliquoted into a 2 mm electroporation cuvette. Prior to nsPEF exposure, a resting temperature (° C.) of the NaCl solution was obtained by inserting a ‘K’ thermocouple probe into the solution and recorded using the N1 temperature logger software from National Instruments. Subsequently, the NaCl solution was exposed to the above mentioned nsPEF exposure parameters. Immediately following exposure, the probe was re-inserted into the NaCl solution and recorded for any change in temperature. Change in temperature (ΔT) was obtained by subtracting the pre-exposure temperature from the post exposure temperature.

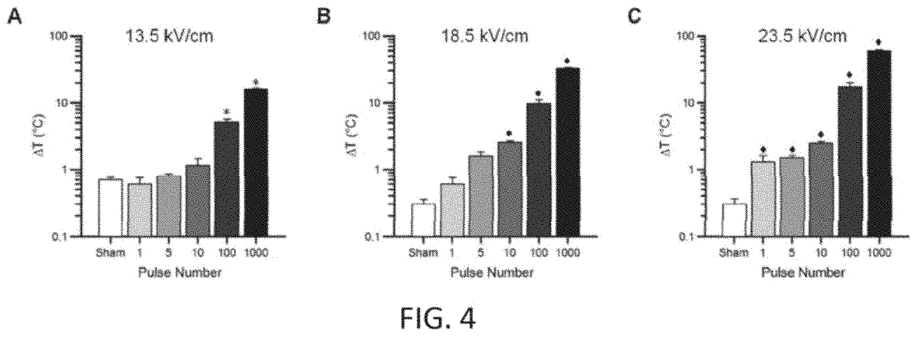

FIG. 4 illustrates three graphical representations of thermal response. In total, 3 cell suspensions of 120 μl each were pipetted into 3-200 μl PCR tubes (BioRad TCS0803), capped (BioRad TLS0801) and placed into an Eppendorf Mastercycler gradient thermocycler. A resting temperature (° C.) of the cell solution was obtained by inserting the thermocouple into a fourth PCR tube containing only 0.85% NaCl solution and recorded as mentioned above. The thermocycler was then set to the ΔT displayed in FIG. 4. Thermal exposure time was equivalent to 1 Hz (˜1 pulse/sec) and was initiated once the final temperature was reached.

Viability was assessed immediately following one set of exposures (i.e. nsPEF or thermal response for: sham, 1, 5, 10, 100 and 1000 pulses). Protocol was followed according to manufacturer's fluorescence microplate reader protocol (7.1-7.7 & 9.1-9.3).

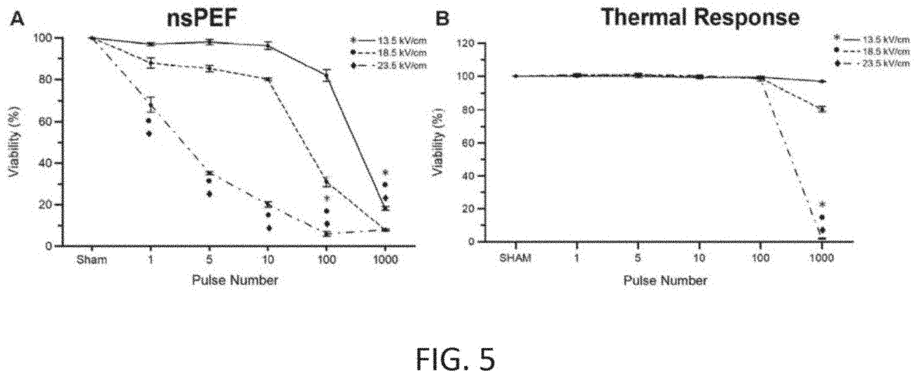

FIG. 5 shows L. acidophilus nsPEF and Thermal Response Viability Curves. Viability curves following 600 ns PEF exposure (A) or thermal exposure (B) corresponding to the ΔT observed in FIG. 1 for 0 (sham), 1, 5, 10, 100 or 1000 pulses.

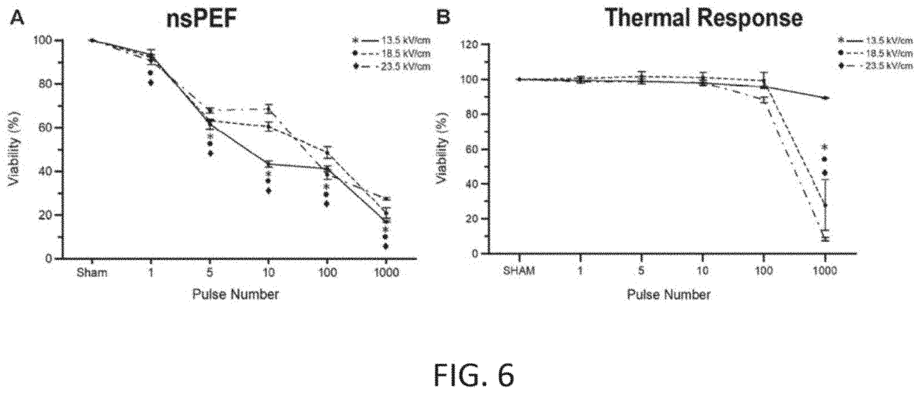

FIG. 6 shows K. pneumoniae nsPEF and Thermal Response Viability Curves. Viability curves following 600 nsPEF exposure (A) or thermal exposure (B) corresponding to the ΔT observed in FIG. 1 for 0 (sham), 1, 5 10, 100 or 1000 pulses.

Statistical Analysis: All experimental conditions were performed in total 3 times, n=3, and were conducted on independent days. Values represent the mean of 3 replicates. Error bars represent standard error. Results that are statistically significantly different from the sham are indicted by an asterisk (*), enclosed circle (•) or enclosed diamond (♦).

The conclusions include: nsPEF affects bacterial cell viability; viability is affected by pulse number and voltage; any heat generated by the nsPEF does not significantly affect cell viability (until at least 1000 pulses is delivered); and generally, K pneumoniae (gram negative) is more susceptible to the effects of nsPEF than L. acidophilus (gram positive).

Various materials can be used to construct the capsule or probe as long as they are able to produce the specific electrical parameters that will be utilized to create pores in bacterial cell wall/membranes and can withstand the pH requirements needed to pass through the gut environment.

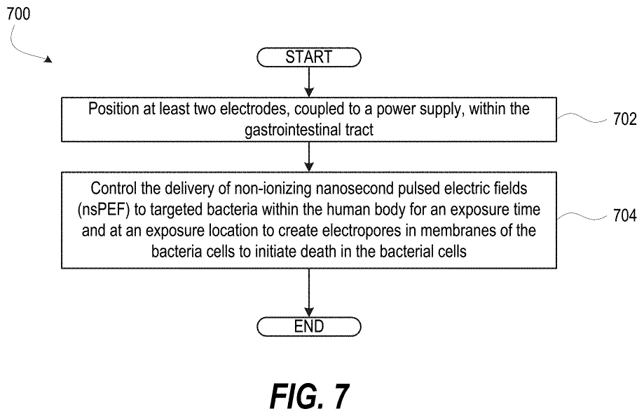

FIG. 7 shows a flow diagram of a method 700 for in-vivo treatment of bacterial cells in the human body. In one or more embodiments, method 700 includes positioning at least two electrodes, coupled to a power supply, within the gastrointestinal tract (block 702). Method 700 includes controlling the delivery of non-ionizing nanosecond pulsed electric fields (nsPEF) to targeted bacteria within the human body for an exposure time and at an exposure location to create electropores in membranes of the bacteria cells to initiate death in the bacterial cells (block 704). Then method 700 ends.

An object of the embodiments of the invention is to treat bacterial infections that are present within the GI system. To combat infections in this location the embodiments include a pill format, or a medical probe, that will allow for access to these locations as well as an ease of use for the patient. However, this type of directed energy may also be effective at treating bacterial infections throughout other locations in the body. As such, the mode of delivery may need various modifications to treat these other locations.

In addition to humans, the embodiments of the invention may be used to treat animals with bacterial induced GI ailments.

The above description provides specific details, such as material types and processing conditions to provide a thorough description of example embodiments. However, a person of ordinary skill in the art would understand that the embodiments may be practiced without using these specific details.

Some of the illustrative aspects of the present invention may be advantageous in solving the problems herein described and other problems not discussed which are discoverable by a skilled artisan. While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

1. An ingestible capsule for in-vivo treatment of bacterial cells in the human body comprising:
a capsule body carrying at least two electrodes;
a power supply; and
a controller, coupled to the power supply and the at least two electrodes, and configured to control the delivery of non-ionizing nanosecond pulsed electric fields (nsPEF) to targeted bacteria within the human body for an exposure time and at an exposure location.

2. A medical probe for in-vivo treatment of bacterial cells in the human body comprising:
a scope carrying at least two electrodes at a distal end thereof;
a power supply; and
a controller, coupled to the power supply and the at least two electrodes, and configured to control the delivery of non-ionizing nanosecond pulsed electric fields (nsPEF) to targeted bacteria within the human body for an exposure time and at an exposure location.

3. A method for in-vivo treatment of bacterial cells in the human body comprising:
positioning at least two electrodes, coupled to a power supply, within the gastrointestinal tract; and
controlling the delivery of non-ionizing nanosecond pulsed electric fields (nsPEF) to targeted bacteria within the human body for an exposure time and at an exposure location to create electropores in membranes of the bacteria cells to initiate death in the bacterial cells.
